(19) United States
(12) Patent Application Publication
(10) Pub. No.: US 2022/0370791 A1
(43) Pub. Date: Nov. 24, 2022

(54) BMP9 MODULATION

(71) Applicant: Leonhardt Ventures LLC, Mission Viejo, CA (US)

(72) Inventors: Howard J. Leonhardt, Mission Viejo, CA (US); Jorge Genovese, Irvine, CA (US)

(21) Appl. No.: 17/663,465

(22) Filed: May 16, 2022

Related U.S. Application Data
(60) Provisional application No. 63/189,586, filed on May 17, 2021.

(51) Int. Cl. A61N 1/05

(52) U.S. Cl. CPC A61N 1/0548

(57) ABSTRACT

Described is a low voltage, pulsed electrical stimulation device for modulating expression of BMP9 protein(s) by cellular tissues.

### CROSS-REFERENCE TO RELATED APPLICATION

This application claims the benefit under 35 U.S.C. § 119(e) of U.S. Provisional Patent Application Ser. No. 63/189,586, filed May 17, 2021, the disclosure of which is hereby incorporated herein in its entirety by this reference.

### TECHNICAL FIELD

The application relates generally to the field of medical devices and associated treatments, and more specifically to precise bioelectrical stimulation of a subject's tissue. More specifically, the application relates to a device having programmed bioelectric signaling sequences, and associated methods for the controlled modulation of BMP9 via precise bioelectrical signaling sequences useful in, for example, orthodontic procedures.

### BACKGROUND

Hustedt & Blizzard. (2014) infra, described that bone morphogenetic proteins (BMPs) have been in use in spinal surgery since 2002. These proteins are members of the TGF-beta superfamily and guide mesenchymal stem cells to differentiate into osteoblasts to form bone in targeted tissues. Since the first commercial BMP became available in 2002, a host of research has supported this use of BMPs and they have been rapidly incorporated in spinal surgeries in the United States. When bound to transmembrane receptors on mesenchymal stem cells, BMPs induce differentiation into osteoprogenitor cells and form new bone.

Bone morphogenetic protein 9 (BMP9) also known as Growth differentiation factor 2 (GDF2) is a protein that in humans is encoded by the GDF2 gene. BMP9 belongs to the transforming growth factor beta superfamily. BMP9 is one of the most potent BMPs to induce orthotopic bone formation in vivo. BMP3, a blocker of most BMPs does not seem to affect BMP9.

Khorsand et al. et al. (2017) infra, described a comparative study of the bone regenerative effect of chemically modified RNA encoding BMP-2 or BMP-9, wherein the connectivity density of the regenerated bone was higher (2-fold-higher) in the group that received BMP-9-cmRNA compared to BMP-2-cmRNA.

### BRIEF SUMMARY

Described herein is a bioelectric stimulator programmed to produce at least one bioelectric signal that modulates (upregulates or downregulates) the expression and/or release of BMP9 in a mammalian target tissue.

In certain embodiments, the bioelectric stimulator is programmed to produce at least one bioelectric signal that upregulates the expression of BMP9 in the target tissue. In certain embodiments, the bioelectric stimulator is programmed to produce at least one bioelectric signal that downregulates the expression of BMP9 in the target tissue. Down regulation of BMP9 is useful for loosening up bone to reform dental arches, teeth, etc., while upregulation is good to build bone to hold teeth in place after movement or re-alignment.

In certain embodiments, described is a low voltage bioelectric stimulator programmed to produce at least one bioelectric signal that upregulates or downregulates BMP9 in a target tissue.

In certain embodiments, the bioelectric stimulator produces a bioelectric signal that upregulates BMP9 in the target tissue. Described is that such BMP9 upregulating bioelectric signals are, e.g., 100 Hz and/or 300 Hz.

In certain embodiments, the bioelectric stimulator is programmed to produce a bioelectric signal that downregulates BMP9 in the target tissue. Described is that such a BMP9 downregulating bioelectric signal is, e.g., 400 Hz.

Signals can be delivered using a constant current or a constant voltage delivery method. Constant current delivery typically ranges from 100 μA to 50 mA. If cellular body tissue such as the subject's skin is contacted, bioelectric signals are typically allowed to increase to the point that a somatosensory response is reported by the patient. Constant voltage delivery would typically range from 1 mV to 20 V/cm. These ranges can vary, dependent upon the resistance of the cellular tissue to be treated. In certain embodiments, the bioelectric signal may be measured at the level of the cell being treated. In certain embodiments, the bioelectric signal may be measured three millimeters in the patient's cellular tissue.

In certain embodiments, the bioelectric stimulator is programmed to produce at least one further bioelectric signal, which aids the subject.

The described bioelectric stimulator may be used to stimulate tissue of a subject, the method comprising: connecting the bioelectric stimulator to the target tissue of the subject, and actuating the bioelectric stimulator to produce the programmed bioelectric signal(s).

Typically, the subject or patient's treated cellular tissue is dental gum, bone, or the dental arch of a subject in need thereof.

While not intending to be bound by theory, the described system utilizes precise bioelectric signaling that appear to communicate with DNA and cell membranes within stimulated tissues of the subject to cause the stimulated cells to produce high volumes of BMP9 protein(s). Useful indications include bone healing and bone integration (e.g., for use with an implant such as a dental implant).

BMP9 expression may be regulated in any tissue or bone including adipose tissue, derived stromal fraction, amniotic membranes, amniotic secretome, platelet rich fibrin (“PRF”), and other cells and tissues.

### BRIEF DESCRIPTION OF THE DRAWINGS

FIG. 1 depicts a programmed bioelectric stimulator for delivery to a subject connected to multiple soft conductive electrode pads.

FIG. 2 is a graph depicting the modulation of BMP9 as described in the Example hereof.

### DETAILED DESCRIPTION

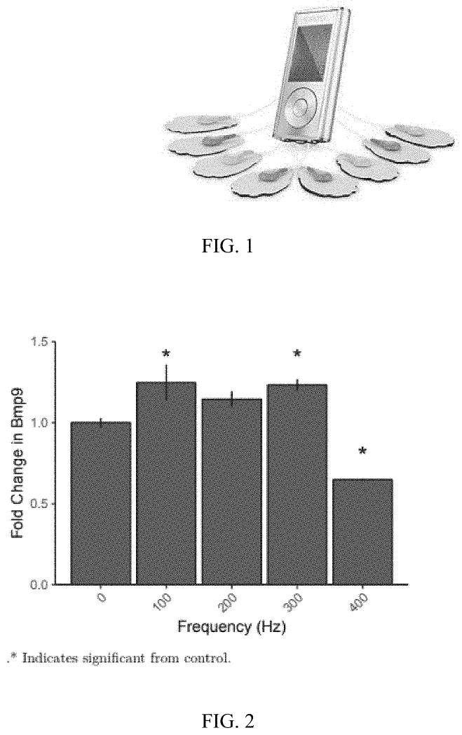

Referring now to FIG. 1, depicted is a biostimulator for use in the treatment of a, for example, human subject. A bioelectric stimulator typically includes a low voltage electrical signal generator programmed to produce the selected bioelectric signal(s) associated with electrodes for delivering the bioelectric signal(s) to the patient's cellular tissue.

A micro voltage signal generator for use herein may be produced utilizing the same techniques to produce a standard heart pacemaker well known to a person of ordinary skill in the art. An exemplary microvoltage generator is available from Mettler Electronics Corp. of Anaheim, Calif., US or HTM Electronica of Amparo, BR. The leading pacemaker manufacturers are Medtronic, Boston Scientific Guidant, Abbott St. Jude, BioTronik and Sorin Biomedica.

Construction of the electric signal generators and pacemakers, are known in the art and can be obtained from OEM suppliers as well as their accompanying chargers and programmers. The electric signal generators are programmed to produce specific signals to lead to specific protein expressions at precisely the right time for, e.g., optimal treatment or regeneration.

The bioelectric stimulator of FIG. 1 is depicted as a programmed electric signal generator with leads connecting it to multiple soft conductive electrode pads. Electrodes may be used to deliver a bioelectric signal to the subject.

When the patient's treated cellular tissue is dental gum, bone, or dental arch, the electrodes may be placed for administration to the patient using the orthodontic devices described in U.S. Pat. No. 10,695,563 to Leonhardt et al. (Jun. 30, 2020) for “Orthodontic Treatment” or US 20200330753 Al to Leonhardt et al. for “Orthodontic treatment,” published on Oct. 22, 2020, the contents of each of which is incorporated herein by this reference.

The biostimulator is actuated and runs through programmed signals to modulate the production of a bioelectric signal or signals that can induce a subject to increase or decrease the expression of, e.g., BMP9 protein for delivery to the subject.

Typical subjects to be treated are mammals such as humans.

In certain embodiments, the bioelectric stimulator is programmed to produce further bioelectric signals, such as those disclosed in U.S. Pat. No. 10,960,206 to Leonhardt et al. for “Bioelectric Stimulator” (Mar. 20, 2021), the contents of the entirety of which are incorporated herein by this reference. Described therein are bioelectric signals to induce expression by cellular tissue of osteoprotegerin or “OPG,” RANKL, SDF-1, PDGF, a signal for stem cell homing, PDGF, different signals for stem cell proliferation, activin-B, EGF, IGF-1, tropoelastin, VEGF, follistatin, HGF, and any combination thereof. Other useful bioelectric signals for use herein are described in the incorporated U.S. Pat. No. 10,695,563 to Leonhardt et al. and US 20200330753 A1 to Leonhardt et al.

The invention is further described with the aid of the following illustrative Example.

### EXAMPLES

### Exampe —Controlling Expression and/or Release of BMP9

Purpose: The purpose of this Example was to analyze the effects of bioelectric signal stimulation on BMP9 in platelet rich fibrin (“PRF”) stimulated at 100 Hz, 200 Hz, 300 Hz, and 400 Hz at 1 V for 30 minutes and compare its expression against a control (unstimulated) condition using the basic enzyme-linked immunosorbent assay (ELISA).

Electrical Signals

100 Hz, 1 V

200 Hz, 1 V

300 Hz, 1 V

400 Hz, 1 V

Target Protein: BMP9

Methods: Human blood without anticoagulants was collected and immediately centrifuged at 900 rpm for 5 minutes. PRF was collected and equally plated in a 6-well dish with 1 mL/per well DMEM (10% FBS). Samples were stimulated with a RIGOL biostimulator (Suzhou, China) at the described frequencies for 30 minutes and the other three wells were control (unstimulated) samples.

Post-stimulation, media was collected and Human BMP9 was quantified using QUANTIKINE® ELISA kits according to the manufacturer's instructions (R&D Systems, Minneapolis, Minn., US) on a Enspire 2300 multilabel microplate reader (Perkin Elmer, Wallac Oy, Turku, Finland).

Conclusions: After adjustments (4 tests), post hoc tests showed 100 Hz and 300 Hz increased expression of BMP9 and 400 Hz decreased expression of BMP9.

In summary, these data (shown graphically in FIG. 2) show that bioelectric signal treatment can be used to increase or decrease BMP9 protein concentration in platelet-rich fibrin and the sensitivity of the assay used.

Results

Summary: Fold Change

| ## | | Frequency | N | FoldChange | sd | se | ci |
|---|---|---|---|---|---|---|---|
| ## | 1 | 0 | 4 | 1.00 | 0.06 | 0.03 | 0.09 |
| ## | 2 | 100 | 4 | 1.25 | 0.22 | 0.11 | 0.35 |
| ## | 3 | 200 | 4 | 1.15 | 0.09 | 0.05 | 0.15 |
| ## | 4 | 300 | 4 | 1.23 | 0.07 | 0.03 | 0.11 |
| ## | 5 | 400 | 4 | 0.65 | 0.01 | 0.00 | 0.01 |

ANOVA Fold Change

| | |
|---|---|
| ## | [1] “F-statistic: 3.81 on 1 and 18 DF, p-value: 0.06667” |

Post Hoc Test

| ## | Analysis of Variance Table | | | | | | |
|---|---|---|---|---|---|---|---|
| ## | | | | | | | |
| ## | Response: FoldChange | | | | | | |
| ## | | Df | Sum Sq | Mean Sq | F value | Pr(>F) | |
| ## | FrequencyFactor | 4 | 0.98186 | 0.24546 | 18.809 | 1.037e−05 | *** |
| ## | Residuals | 15 | 0.19575 | 0.01305 | | | |

| | |
|---|---|
| ## | ~~~ |
| ## | Signif. codes: 0 ‘***’ 0.001 ‘**’ 0.01 ‘*’ 0.05 ‘.’ 0.1 ‘’ 1 |

| ## | [1] “Comparisons” | | | | | | | |
|---|---|---|---|---|---|---|---|---|
| ## | | contrast | estimate | SE | df | t. ratio | p. value | p. value. adj |
| ## | 1 | 0-100 | −0.248 | 0.081 | 15 | −3.072 | 0.008 | 0.015 |
| ## | 2 | 0-200 | −0.146 | 0.081 | 15 | −1.804 | 0.091 | 0.130 |
| ## | 3 | 0-300 | −0.233 | 0.081 | 15 | −2.891 | 0.011 | 0.019 |
| ## | 4 | 0-400 | 0.351 | 0.081 | 15 | 4.348 | 0.001 | 0.001 |
| ## | 5 | 100-200 | 0.102 | 0.081 | 15 | 1.268 | 0.224 | 0.280 |
| ## | 6 | 100-300 | 0.015 | 0.081 | 15 | 0.182 | 0.858 | 0.858 |
| ## | 7 | 100-400 | 0.599 | 0.081 | 15 | 7.420 | 0.000 | 0.000 |
| ## | 8 | 200-300 | −0.088 | 0.081 | 15 | −1.086 | 0.294 | 0.327 |
| ## | 9 | 200-400 | 0.497 | 0.081 | 15 | 6.152 | 0.000 | 0.000 |
| ## | 10 | 300-400 | 0.585 | 0.081 | 15 | 7.239 | 0.000 | 0.000 |

### REFERENCES

(The contents of the entirety of each of which is incorporated herein by this reference.)

Fujioka-Kobayashi et al. “Absorbable collagen sponges loaded with recombinant bone morphogenetic protein 9 induces greater osteoblast differentiation when compared to bone morphogenetic protein 2″ Clin Exp Dent Res 2017; 3:32-40.

Hustedt, Joshua W, and Daniel J Blizzard. “The controversy surrounding bone morphogenetic proteins in the spine: a review of current research.” The Yale Journal of Biology and Medicine vol. 87,4 549-61.12 Dec. 2014

Khorsand, Behnoush et al. “A Comparative Study of the Bone Regenerative Effect of Chemically Modified RNA Encoding BMP-2 or BMP-9.” The AAPS Journal vol. 19,2 (2017): 438-446. doi:10.1208/s12248-016-0034-8.

Liu et al. “BMP9 is a potential therapeutic agent for use in oral and maxillofacial bone tissue engineering” Biochem Soc Trans. 2020 Jun 30;48(3):1269-1285. doi: 10.1042/B5T20200376.

U.S. Pat. No. 10,695,563 to Leonhardt et al. (Jun. 30, 2020) for “Orthodontic Treatment”.

U.S. Pat. No. 10,960,206 to Leonhardt et al. for “Bioelectric Stimulator” (Mar. 20, 2021).

US 20200330753 A1 to Leonhardt et al. for “Orthodontic treatment,” published on Oct. 22, 2020.

What is claimed is:

1. A bioelectric stimulator programmed to produce a bioelectric signal that modulates expression and/or release of bone morphogenetic protein 9 (BMP9) in a cell.

2. The bioelectric stimulator of claim 1, wherein the produced bioelectric signal upregulates the expression and/or release of BMP9 in the cell.

3. The bioelectric stimulator of claim 2, wherein the bioelectric signal is 100 Hz or 300 Hz.

4. The bioelectric stimulator of claim 3, wherein the produced bioelectric signal is 100 Hz.

5. The bioelectric stimulator of claim 3, wherein the produced bioelectric signal is 300 Hz.

6. The bioelectric stimulator of claim 1, wherein the programmed bioelectric signal downregulates the expression and/or release of BMP9 in the cell.

7. The bioelectric stimulator of claim 6, wherein the produced bioelectric signal is 400 Hz.

8. The bioelectric stimulator of claim 1, wherein the bioelectric stimulator is programmed to produce a plurality of bioelectric signals.

9. A method of using the bioelectric stimulator of claim 1, to stimulate cellular tissue, the method comprising:
connecting the bioelectric stimulator to the cellular tissue, and
actuating the bioelectric stimulator to produce the programmed bioelectric signal(s) so as to modulate expression and/or release of bone morphogenetic protein 9 (BMP9) in the cellular tissue.

10. The method according to claim 9, wherein the tissue is selected from the group consisting of bone, dental arch, dental gum tissue, adipose tissue, derived stromal fraction, amniotic membranes, amniotic secretome, platelet rich fibrin (“PRF”), and any combination(s) thereof.

11. The method according to claim 9, wherein the bioelectric signal is selected from the group consisting of 100 Hz (within 15%), 300 Hz (within 15%), 400 Hz (within 15%), and a combination thereof.

12. A method of treating a cell, the method comprising:
stimulating the cell to express and/or release of bone morphogenetic protein 9 (BMP9) by applying a bioelectric signal to the cell, wherein the bioelectric signal comprises, within 15%, a biphasic pulse of 100 Hz and/or 300 Hz.

13. The method according to claim 12, wherein the bioelectric signal is 100 Hz.

14. The method according to claim 12, wherein the bioelectric signal is 300 Hz.

15. A method of treating a cell, the method comprising:
stimulating the cell to downregulate expression and/or release of bone morphogenetic protein 9 (BMP9) by applying a bioelectric signal to the cell, wherein the bioelectric signal is, within 15%, a biphasic pulse of 400 Hz.
